(19) United States
(12) Patent Application Publication
(10) Pub. No.: US 2023/0293193 A1
(43) Pub. Date: Sep. 21, 2023

(54) CLOTLYSIS FROM ULTRASONIC HARMONIC EXCITATION

(71) Applicant: Open Water Internet Inc., San Francisco, CA (US)

(72) Inventors: Achal Singh Achrol, Highland, CA (US); Soren Konecky, Alameda, CA (US); Christopher R. Bawiec, San Francisco, CA (US); Brad A. Hartl, San Mateo, CA (US); Ulas Taskin, Delft (NL); Sarah Barreto Ornellas, San Francisco, CA (US)

(21) Appl. No.: 18/121,474

(22) Filed: Mar. 14, 2023

Related U.S. Application Data
(60) Provisional application No. 63/321,060, filed on Mar. 17, 2022.

(51) Int. Cl. A61B 17/22

(52) U.S. Cl. CPC A61B 17/22004; A61B 2090/502

(57) ABSTRACT

A wearable device includes one or more ultrasonic emitters disposed to direct a therapeutic ultrasound signal into tissue. The therapeutic ultrasound signal has a resonant ultrasound frequency to harmonically excite blood clots to liquify the blood clot to become soluble to blood flowing through the head.

### CROSS-REFERENCE TO RELATED APPLICATIONS

This application claims priority to U.S. provisional Application No. 63/321,060 filed Mar. 17, 2022, which is hereby incorporated by reference.

### TECHNICAL FIELD

This disclosure relates generally to medical therapies, and in particular to ultrasound therapies.

### BACKGROUND INFORMATION

A stroke occurs when blood supply to the brain is interrupted or reduced. This lack of blood flow may prevent the brain from receiving sufficient oxygen leading to death or damage of brain cells. Strokes may be caused by plaque buildup in arteries. Depending on the severity of the stroke, patients may experience numbness, partial paralysis, weakness in the face/legs/arms, difficulty speaking, difficulty processing speech, dizziness, and/or loss of balance. Treatments for stroke may involve surgery, medication, or rehabilitation therapies. The earlier a stroke is noticed and treated, generally the better the outcome for the patient.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified.

FIG. 1 illustrates a clotlysis therapy system including processing logic and one or more ultrasound emitters, in accordance with aspects of the disclosure.

FIG. 2 illustrates an example flow chart illustrating an example clotlysis process, in accordance with aspects of the disclosure.

FIG. 3 illustrates a head wearable device including a plurality of ultrasound emitter locations, in accordance with aspects of the disclosure.

FIG. 4 illustrates an example clotlysis system that includes a head mountable wearable communicatively coupled to a mobile device, in accordance with aspects of the disclosure.

### DETAILED DESCRIPTION

Embodiments of systems, devices, and methods of clotlysis from ultrasonic harmonic excitation are described herein. In the following description, numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In aspects of this disclosure, visible light may be defined as having a wavelength range of approximately 380 nm-700 nm. Non-visible light may be defined as light having wavelengths that are outside the visible light range, such as ultraviolet light and infrared light. Infrared light having a wavelength range of approximately 700 nm-1 mm includes near-infrared light. In aspects of this disclosure, near-infrared light may be defined as having a wavelength range of approximately 700 nm-1.6 μm.

Throughout this specification, several terms of art are used. These terms are to take on their ordinary meaning in the art from which they come, unless specifically defined herein or the context of their use would clearly suggest otherwise.

A stroke may occur when a blood clot blocks or narrows an artery leading to the brain. Recently, ultrasound has been used for transcranial therapies including clot breakdown. These prior techniques use ultrasonic frequencies of around 2 MHz or greater which are thought to loosen fibrin strands in blood structures. To direct the 2 MHz ultrasound signal into a head of a user, the ultrasound emitter or transducer would be placed in the temple area or at the junction of the ear and jawbone so that the 2 MHz signal could penetrate through to the brain. These preferred locations allowed the high frequency 2 MHz ultrasound signal to be directed through the thinnest part of the skull (temple area) or just beneath the skull. However, this placement of the ultrasound emitter/transducer limits the area of the brain where the ultrasonic therapy can be directed.

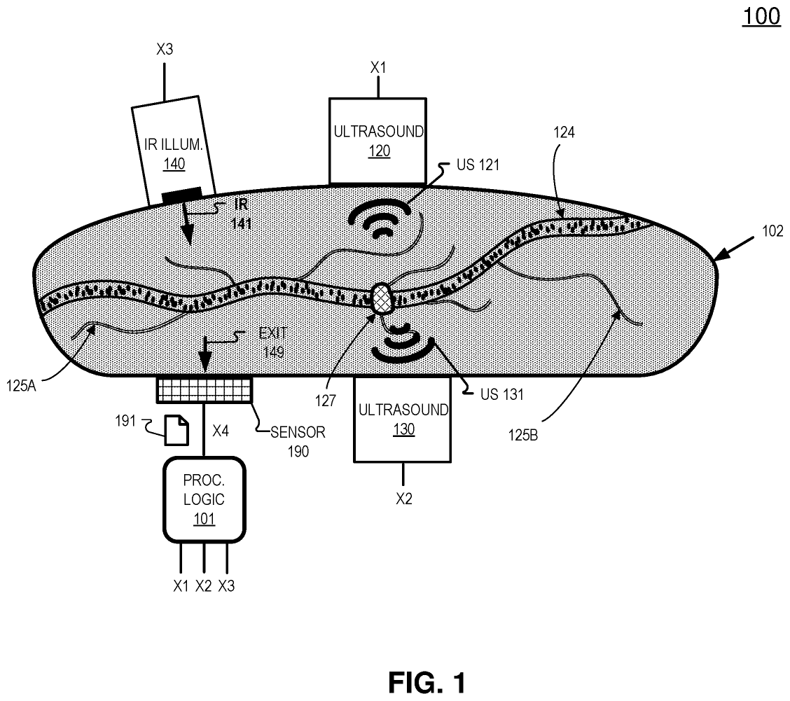

Systems, devices, and methods of this disclosure include ultrasound transducers directing therapeutic ultrasound signals to blood clots where the therapeutic ultrasound signal has a resonant ultrasound frequency to harmonically excite the blood clots to liquify the blood clot to become soluble to blood flowing through the head of a patient. Implementations of the disclosure may also be used to harmonically excite (and liquify) blood clots in other portions of the body besides the head. The resonant ultrasound frequency is less than 1 MHz. In some implementations, the resonant ultrasound frequency is between 50 kHz and 700 kHz. These lower ultrasonic frequencies liquify blood clots in the same way that an earthquake may liquify soil. In the context of blood clots formed in the head/brain, the lower frequency of the ultrasonic signal allows the therapeutic ultrasound signals to penetrate through thicker parts of the skull to the brain. Thus, the ultrasonic emitters can be placed in a variety of locations on the head rather than being limited in placement to the temple region. These and other implementations will be described in FIGS. 1-4.

FIG. 1 illustrates a clotlysis therapy system 100 including processing logic 101 and one or more ultrasound emitters, in accordance with aspects of the disclosure. In the particular implementation of FIG. 1, system 100 includes ultrasonic emitter 120 configured to emit therapeutic ultrasonic signal 121 and ultrasonic emitter 130 configured to emit therapeutic ultrasonic signal 131. Ultrasonic emitters 120 and 130 may be included in ultrasonic transducers, in some implementations. Ultrasonic emitters 120 and 130 may be steerable ultrasound emitters, in some implementations. For example, an array of ultrasonic emitters may be driven to steer their therapeutic ultrasonic signal by utilizing phase delays to affect the direction of the therapeutic ultrasonic signal. This functionality may be referred to as “beam forming.” System 100 may include any number of ultrasonic emitters or transducers to deliver therapeutic ultrasound signals to tissue.

Some or all of the components of system 100 may be included in a wearable device or article that is configured to have the ultrasonic emitters contact the skin of a patient so that the therapeutic ultrasonic signal 121 may propagate into the tissue 102. The wearable device may be configured to be secured to a head, arm, or other body part. Tissue 102 may represent a brain. Tissue 102 includes an artery 124 and blood vessels 125 (e.g. blood vessels 125A and 125B). A clot 127 has narrowed, occluded or blocked artery 124, in FIG. 1.

Processing logic 101 is configured to drive ultrasound emitter 120 via communication channel X1 and configured to drive ultrasound emitter 130 via communication channel X2. In an implementation, processing logic 101 is configured to drive the one or more ultrasonic transducers to direct a therapeutic ultrasound signal (e.g. signal 121 or 131) into tissue 102 brought into contact with the ultrasonic transducers. The therapeutic ultrasound signal(s) have a resonant ultrasound frequency to harmonically excite and liquify clot 127. Liquifying clot 127 allows clot 127 to become soluble and reduce the size of clot 127 as blood clot 127 dissolves into the blood stream of artery 124.

Processing logic 101 may drive one or more ultrasonic emitters to emit a therapeutic ultrasonic signal at a resonant ultrasound frequency below 1 MHz to harmonically excite clot 127 and therefore cause clotlysis of clot 127. In some implementations, the resonant ultrasound frequency is between 50 kHz and 700 kHz. In some implementations, the resonant ultrasound frequency is between 70 kHz and 500 kHz. In some implementations, the resonant ultrasound frequency is between 100 kHz and 200 kHz.

In some implementations of system 100, an infrared illuminator such as an infrared laser or infrared LED is included. Processing logic 101 may be configured to activate (turn on) and/or deactivate (turn off) infrared illuminator 140 by way of communication channel X3. Infrared illuminator 140 is configured to emit infrared light 141 into tissue 102. Infrared light 141 may be near-infrared light. Infrared light 141 may be centered around 850 nm or 940 nm, for example. Infrared light 141 may be coherent light. Infrared light 141 may have a narrow beamwidth.

System 100 may also include a light sensor 190 to receive an infrared exit signal 149. Infrared light 141 will propagate and scatter within tissue 102. Infrared exit signal 149 is a portion of the infrared light 141 exiting tissue 102. Sensor 190 may be a photodiode, a photodiode array, or a complementary metal oxide semiconductor (CMOS) image sensor, for example. Sensor 190 may include an infrared filter that passes a narrow beamwidth of light 141 (and consequently exit signal 149) while rejecting all other wavelengths. Sensor 190 generates an infrared exit signal measurement 191 that is provided to processing logic 101 via communication channel X4, in FIG. 1. Processing logic 101 may be configured to modulate one or more of therapeutic ultrasound signal(s) 121 and 131 in response to the infrared exit signal measurement 191. In an implementation, processing logic 101 is configured to modulate a resonant ultrasound frequency of the therapeutic ultrasound signal in response to infrared exit signal measurement 191. Infrared exit signal measurement 191 may be an analog or digital value or an image.

In an implementation, processing logic 101 is further configured to measure a laser speckle value in the infrared exit signal and modulate the therapeutic ultrasound signal 121/131 in response to the infrared exit signal 149 includes modulating the therapeutic ultrasound signal based at least in part on the laser speckle value. The laser speckle value of the brain may be indicative of blood flow, blood volume, and/or blood oxygenation data, for example. Therefore, system 100 may receive some feedback about the effectiveness of the harmonic excitation of clot 127 by analyzing infrared exit signal measurement 191.

In implementations where laser speckle is analyzed by processing logic 101, coherent light interference in an image (e.g. an image included in infrared exit signal measurement 191) may be manifest or captured as speckles, which include bright and dark spots of one or more pixels in an image. Dark pixels are pixels that have a lower pixel value than surrounding pixels and/or than the average pixel value of an image. Bright pixels are pixels that have a higher pixel value than surrounding pixels and/or than the average pixel value of an image. Quantities of speckles, and therefore coherent light interference, in an image may be detected using the standard deviation of all of the pixels of an image. More specifically, speckle contrast may be determined by dividing the standard deviation of the pixel values of an image by the mean of the pixel values of an image (i.e., std/mean). The speckle contrast of an image is compared to one or more data models that map the speckle contrast to quantities of blood flowing through a tissue sample, in an embodiment. Blood characteristics may include the quantity of blood flowing through an area, the velocity of the blood, and may also include the concentration and oxygenation levels of hemoglobin. Some blood characteristics are blood flow characteristics, and blood flow characteristics may include the quantity of blood flowing through a region of tissue and the velocity of blood flowing through a region of tissue. Some blood characteristics may be independent or less dependent on blood flow, and these blood characteristics may include the concentration and oxygenation levels of hemoglobin.

In implementations of the disclosure, modulating the therapeutic ultrasound signals 121 and/or 131 may be in response to the blood flow of the tissue determined by the speckle contrast value determined from analyzing exit signal 149. For example, when a blood flow increases, the intensity of the therapeutic ultrasound signals 121 and/or 131 may be reduced.

In implementations of the disclosure, modulating the therapeutic ultrasound signals 121 and/or 131 may be in response to blood oxygenation levels of the tissue determined by the speckle contrast value determined from analyzing exit signal 149. For example, when blood oxygenation levels increase, the intensity of the therapeutic ultrasound signals 121 and/or 131 may be reduced.

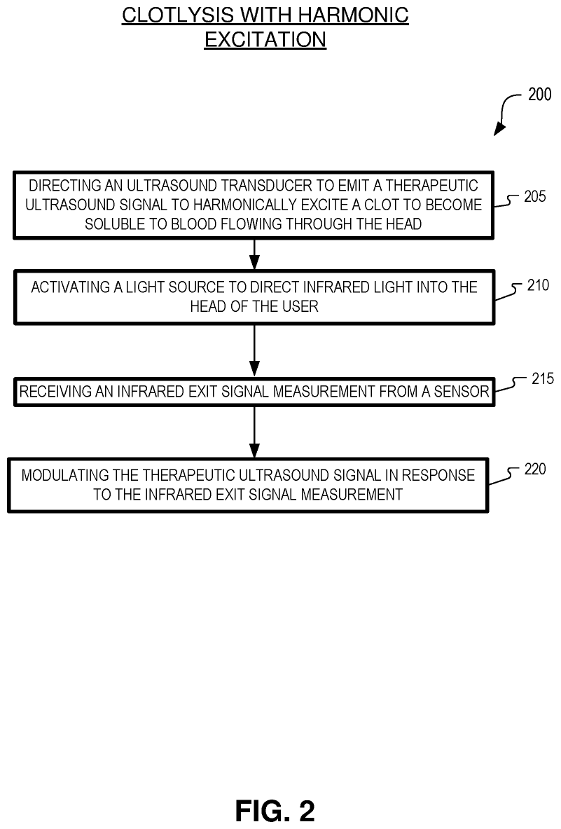

FIG. 2 illustrates an example flow chart illustrating an example process 200, in accordance with implementations of the disclosure. The order in which some or all of the process blocks appear in process 200 should not be deemed limiting. Rather, one of ordinary skill in the art having the benefit of the present disclosure will understand that some of the process blocks may be executed in a variety of orders not illustrated, or even in parallel.

In process block 205, an ultrasound transducer directs a therapeutic ultrasound signal to harmonically excite a clot to become soluble to blood flowing through the head. The resonant ultrasound frequency of the therapeutic ultrasound signal may be less than 1 MHz. In an implementation, the resonant ultrasound frequency of the therapeutic ultrasound signal is between 50 kHz and 700 kHz.

In process block 210, a light source directs infrared light (e.g. light 141) into the head of the user. The light source may be an infrared laser or an infrared LED. In some implementations, the infrared light is near-infrared light.

In process block 215, an infrared exit signal measurement is received from a sensor (e.g. sensor 190). In some implementations, the sensor is a CMOS image sensor. In some implementations, the sensor includes one or more photodiodes.

In process block 220, the therapeutic ultrasound signal is modulated in response to the infrared exit signal measurement. In an implementation, a resonant ultrasound frequency of the therapeutic ultrasound signal is modulated in response to infrared exit signal measurement 191.

In implementations of the disclosure, modulating the therapeutic ultrasound signals may be in response to the blood flow of the tissue determined by the speckle contrast value determined from analyzing the exit signal. For example, when a blood flow increases, the intensity of the therapeutic ultrasound signals may be reduced.

In implementations of the disclosure, modulating the therapeutic ultrasound signals may be in response to blood oxygenation levels of the tissue determined by the speckle contrast value determined from analyzing the exit signal. For example, when blood oxygenation levels increase, the intensity of the therapeutic ultrasound signals may be reduced.

In an implementation, process 200 further includes measuring a laser speckle value in the infrared exit signal where the light source includes an infrared laser. And, modulating the therapeutic ultrasound signal in response to the infrared exit signal includes modulating the therapeutic ultrasound signal based at least in part on the laser speckle value.

In an implementation, one or more ultrasonic transducers are disposed on a head wearable device and disposed outside of a temple-region of a head wearable device.

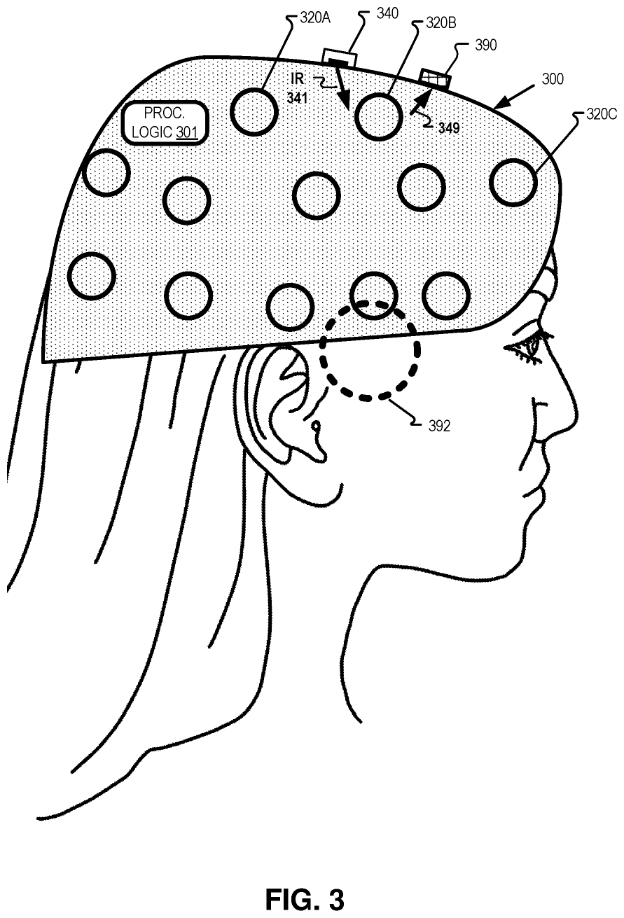

FIG. 3 illustrates a head wearable device 300 including a plurality of ultrasound emitter locations, in accordance with implementations of the disclosure. Head wearable device 300 may include structures or fabric so that head wearable device 300 covers the top of the head like a ski cap would.

The circles 320 in device 300 represent possible locations for ultrasonic emitters/transducers 320 although only ultrasonic emitters/transducers 320A, 320B, and 320C are specifically labeled with reference numerals. Because of the lower frequency (e.g. below 1 MHz) of the therapeutic ultrasonic signal utilized by Applicant, ultrasonic emitters/transducers 320 may be placed outside of the temple region 392 and still deliver therapeutic ultrasound signals to the brain of the user by the lower frequency propagating through the skull. Thus, using the lower frequency enables greater design freedom with respect to placement of the ultrasonic transducers. In contrast, conventional approaches use ultrasound frequencies greater than 2 MHz and are limited to directing an ultrasound signal through the soft tissue near the temple region 392 in order to transmit the higher frequency ultrasound signal through the softer tissue (less bone) of the jaw-ear/skull intersection. Since clots form in different portions of the head (e.g. M1 or internal carotid artery) the design freedom of the transducers to direct the therapeutic ultrasound signal(s) to a greater number of locations is important. The therapeutic ultrasound signal may be directed to the frontal lobe, the parietal lobe, the temporal lobe, the occipital lobe and the cerebellum, for example. Furthermore, adding positions for the ultrasound transducers allows for different angles to direct the therapeutic ultrasound signals and make it more likely that the entire clot can become soluble instead of merely breaking the clot into smaller clots that may then occlude downstream blood vessels.

The illustrated implementation of head wearable device 300 includes processing logic 301, infrared illuminator 340, and light sensor 390. Infrared illuminator 340 is configured to direct infrared light 341 into the head tissue and light sensor 390 is configured to receive infrared exit signal 349 exiting the head tissue. Infrared exit signal 349 is a portion of the infrared light 341 that exits the head tissue. Processing logic 301, infrared illuminator 340, and light sensor 390 may have some or all of the characteristics of processing logic 101, infrared illuminator 140, and light sensor 190, respectively.

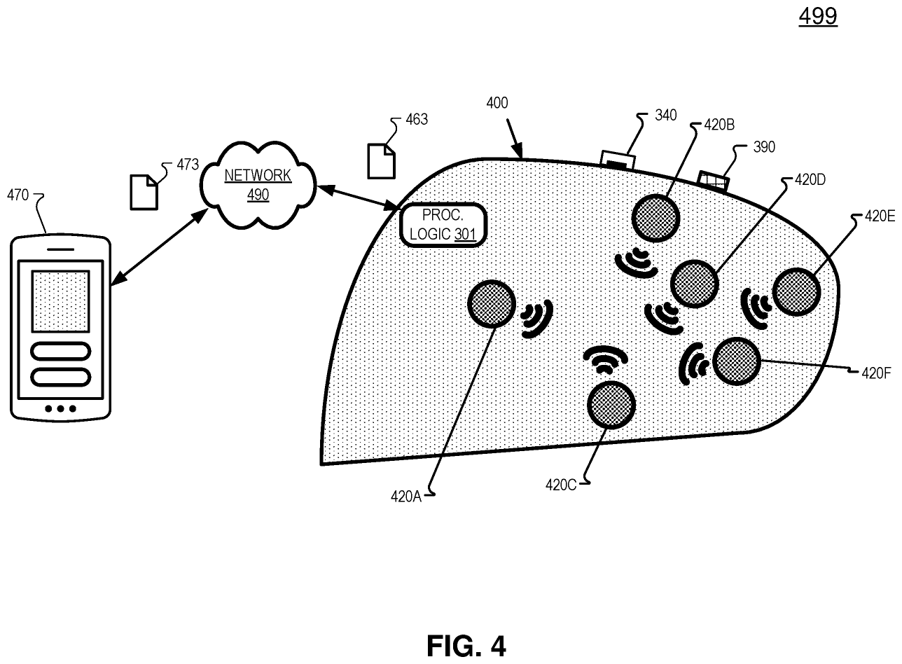

FIG. 4 illustrates an example system 499 that includes a head mountable wearable 400 communicatively coupled to a mobile device 470, in accordance with aspects of the disclosure. In the illustrated implementation, a head mountable wearable 400 includes processing logic 301, infrared illuminator 340, and light sensor 390, and ultrasonic transducers 420A, 420B, 420C, 420D, 420E, and 420F (collectively referred to as ultrasonic transducers 420). The ultrasonic transducers 420 are configured to direct their respective therapeutic ultrasound signals to blood clots at a resonant ultrasound frequency to harmonically excite the clot to become soluble, in accordance with implementations of the disclosure.

In the illustrated implementation of FIG. 4, communications between wearable 400 and mobile device 470 are facilitated by network 490. Network 490 may be wired or wireless. In the illustrated implementation, device 470 may send a command message 473 to wearable 400 via network 490. When processing logic 301 receives command message 473 from device 470, processing logic 301 may initiate a therapeutic ultrasound treatment. In some implementations, processing logic transmits treatment data 463 to device 470 via network 490. Treatment data 463 may include a length of treatment and a resonant ultrasound frequency of the therapeutic ultrasound signals of the treatment, for example. Portions of network 490 may be wired and portions of network 1390 may be wireless. In some implementations, mobile device 470 is a smartphone or a tablet. Device 470 may be a desktop or laptop computer in some implementations. Device 470 may communicate directly with wearable 400, in some implementations.

The term “processing logic” (e.g. 101 or 301) in this disclosure may include one or more processors, microprocessors, multi-core processors, Application-specific integrated circuits (ASIC), and/or Field Programmable Gate Arrays (FPGAs) to execute operations disclosed herein. In some embodiments, memories (not illustrated) are integrated into the processing logic to store instructions to execute operations and/or store data. Processing logic may also include analog or digital circuitry to perform the operations in accordance with embodiments of the disclosure.

A “memory” or “memories” described in this disclosure may include one or more volatile or non-volatile memory architectures. The “memory” or “memories” may be removable and non-removable media implemented in any method or technology for storage of information such as computer-readable instructions, data structures, program modules, or other data. Example memory technologies may include RAM, ROM, EEPROM, flash memory, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device.

Networks may include any network or network system such as, but not limited to, the following: a peer-to-peer network; a Local Area Network (LAN); a Wide Area Network (WAN); a public network, such as the Internet; a private network; a cellular network; a wireless network; a wired network; a wireless and wired combination network; and a satellite network.

Communication channels (e.g. X1, X2, X3, and X4) may include or be routed through one or more wired or wireless communication utilizing IEEE 802.11 protocols, short-range wireless, SPI (Serial Peripheral Interface), I²C (Inter-Integrated Circuit), USB (Universal Serial Port), CAN (Controller Area Network), cellular data protocols (e.g. 3G, 4G, LTE, 5G), optical communication networks, Internet Service Providers (ISPs), a peer-to-peer network, a Local Area Network (LAN), a Wide Area Network (WAN), a public network (e.g. “the Internet”), a private network, a satellite network, or otherwise.

A computing device may include a desktop computer, a laptop computer, a tablet, a phablet, a smartphone, a feature phone, a server computer, or otherwise. A server computer may be located remotely in a data center or be stored locally.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible non-transitory machine-readable storage medium includes any mechanism that provides (i.e., stores) information in a form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A therapy system comprising:
a head wearable device for wearing on or about a head of a user, wherein the head wearable device includes one or more ultrasonic emitters disposed to direct a therapeutic ultrasound signal into the head; and
processing logic configured to drive the one or more ultrasonic emitters, wherein the therapeutic ultrasound signal has a resonant ultrasound frequency to harmonically excite blood clots to liquify the blood clot to become soluble to blood flowing through the head.

2. The therapy system of claim 1, wherein a frequency of the resonant ultrasound frequency is less than 1 MHz.

3. The therapy system of claim 1, wherein a frequency of the resonant ultrasound frequency is between 50 kHz and 700 kHz.

4. The therapy system of claim 1, wherein the one or more ultrasonic emitters are disposed on the head wearable device outside of a temple-region of the head wearable device.

5. The therapy system of claim 1, wherein the head wearable device further includes:
a laser source configured to direct infrared laser light into the head of the user; and
a light sensor configured to receive an infrared exit signal, wherein the infrared exit signal is a portion of the infrared laser light exiting the head of the user,
wherein the processing logic is configured to modulate the therapeutic ultrasound signal in response to the infrared exit signal.

6. The therapy system of claim 5, wherein the processing logic is further configured to measure a laser speckle value in the infrared exit signal, and wherein modulating the therapeutic ultrasound signal in response to the infrared exit signal includes modulating the therapeutic ultrasound signal based at least in part on the laser speckle value.

7. The therapy system of claim 5, wherein the light sensor includes an image sensor.

8. The therapy system of claim 5, wherein the ultrasonic emitters are disposed on the head wearable device to deliver the therapeutic ultrasound signal into a frontal lobe of the head.

9. A wearable device to be worn by a user, the wearable device comprising:
one or more ultrasonic emitters disposed to direct a therapeutic ultrasound signal into tissue of the user; and
processing logic configured to drive the one or more ultrasonic transducers, wherein the therapeutic ultrasound signal has a resonant ultrasound frequency less than 1 MHz to harmonically excite blood clots to liquify the blood clot to become soluble to blood flowing within the user.

10. The wearable device of claim 9, wherein the wearable device further includes:
a laser source configured to direct infrared laser light into tissue of the user; and
a light sensor configured to receive an infrared exit signal, wherein the infrared exit signal is a portion of the infrared laser light exiting the tissue of the user,
wherein the processing logic is configured to modulate the therapeutic ultrasound signal in response to the infrared exit signal.

11. The wearable device of claim 10, wherein the processing logic is further configured to measure a laser speckle value in the infrared exit signal, and wherein modulating the therapeutic ultrasound signal in response to the infrared exit signal includes modulating the therapeutic ultrasound signal based at least in part on the laser speckle value.

12. The wearable device of claim 10, wherein the light sensor includes an image sensor.

13. A non-invasive method of treating vessel occlusion, the non-invasive method comprising:
directing an ultrasound transducer to emit a therapeutic ultrasound signal to harmonically excite a clot to become soluble to blood flowing through a head of a user;
activating a light source to direct infrared light into the head of the user;
receiving an infrared exit signal measurement from a light sensor, wherein the infrared exit signal is a portion of the infrared light exiting the head of the user; and
modulating the therapeutic ultrasound signal in response to the infrared exit signal measurement.

14. The non-invasive method of claim 13 further comprising:
measuring a laser speckle value in the infrared exit signal, the light source including an infrared laser, and wherein modulating the therapeutic ultrasound signal in response to the infrared exit signal includes modulating the therapeutic ultrasound signal based at least in part on the laser speckle value.

15. The non-invasive method of claim 14, wherein the light sensor includes an image sensor.

16. The non-invasive method of claim 13, wherein the ultrasonic transducer configured to emit the therapeutic ultrasound signal is disposed on a head wearable device outside of a temple-region of the head wearable device.

17. The non-invasive method of claim 13, wherein the ultrasonic transducer configured to emit the therapeutic ultrasound signal is disposed on a head wearable device to deliver the therapeutic ultrasound signal into a frontal lobe of the head.

18. The non-invasive method of claim 13, wherein the ultrasonic transducer, the light source, and the light sensor are included in a head mountable wearable.

19. The non-invasive method of claim 13, wherein modulating the therapeutic ultrasound signal includes modulating a resonant ultrasound frequency of the therapeutic ultrasound signal in response to infrared exit signal measurement.

20. The non-invasive method of claim 13, wherein a resonant ultrasound frequency of the therapeutic ultrasound signal is between 50 kHz and 700 kHz.
